(19) United States
(12) Patent Application Publication
(10) Pub. No.: US 2008/0287849 A1
(43) Pub. Date: Nov. 20, 2008

(54) Multi Function knee device

(76) Inventor: Abby Zukowski, Leonia, NJ (US)

Correspondence Address: Abby Zukowski, 540 Nordhoff Drive, Leonia, NJ, 07605, US

(21) Appl. No.: 11/748,546

(22) Filed: May 15, 2007

(51) Int. Cl. A61F 5/00

(52) U.S. Cl. 602/26

(57) ABSTRACT

A knee support device is formed of which possesses a plurality of individual device segments of which comprise the body. Many of the comprising multiple device components of the body may be used independently as separate devices, and are attached, assembled, and joined to form the body. Therapeutic tempered care and maximum support are the main premises to the body. Strategically positioned pockets integrated within the body, along with separate attachable pockets and support pads allow for a maximum and unique tempered therapeutic joint support device.

### BACKGROUND

The present invention relates to an interchangeable hot/cold therapeutic tempered knee support device. Knee devices offered today provide only adequate hot/cold tempered therapy and minimal total joint support due to their lack of diversity in function and design. When such knee devices are utilized by an individual before participating in an activity, the devices offered today do not offer the proper tempered therapy of which provides the options to the wearer of positioning the tempered material within specific strategic locations of which may require therapeutic tempered care. When the devices of prior art are worn by an individual during activity of which requires maximum stability support, and one would require that their knee device of use be able to meet the challenges of combating awkward and unnatural movement which may cause injury to the joint if the maximum support to the majority of the joint is not provided, the individual then would become more susceptible to harmful and painful injuries caused by the threatening pressures to the joint. The devices presently offered do not adequately provide for the needed flexibility of support of which is characterized by the present invention. Oftentimes, after an individual engages in activities of which may prove to be fatiguing and strenuous on the joint, it may then be necessary to treat the joint with hot/cold therapy applied directly to the joint. Prior art devices provide only minimal coverage area protective options, whereas the present invention provides the wearer with multiple optional designated coverage areas of which the wearer may apply hot/cold therapeutic tempered material directly to the specific areas of need on the joint, with this being possible by the functional structural devices and separate attachments of which comprise the device of mention in the specifications. The present invention provides the need for therapeutic tempered applications, maximum support qualities, and also diversity by offering the unique options of separate individual devices of which are interchangeable and together comprise one complete device. The knee devices offered today differ primarily in the areas of design and style, but not in the needed areas of optimal therapeutic and support qualities. As a result, the wearer is offered limited minimal protection of the joint due to the lack of pre-activity therapeutic warm up, maximum support during activity, and post activity therapeutic cool down characteristic options of which are not offered by prior art devices. Prior art devices do not optimally incorporate the combination of therapeutic tempered therapy, maximum stability support and the versatility of interchangeable devices; all of which are premises of which the present invention is structured upon.

### BRIEF DESCRIPTION OF THE DRAWING

The invention may take certain parts and arrangement of parts, a preferred embodiment of which will be described in detail in this specification, and illustrated in accompanying drawings which form a part hereof.

FIG. 1, is a perspective view of the inner cover area of the knee device in accordance with the present invention.

FIG. 2, is a perspective view of the separate individual pocket enclosure, support pad, and fold over flap piece attachments of which affix onto the surface of the knee device.

FIG. 3, is a perspective view of the outer cover area of the knee device in accordance with the present invention.

### BRIEF SUMMARY

The present invention is directed to a joint device specifically targeted to provide therapeutic hot/cold therapy to the joint while simultaneously offering the wearer maximum stability support. The device is truly attachable/detachable in which it is comprised of a plurality of separate individual components of which may be individually worn if so desired. The device attaches and detaches in such an accommodating manner as to provide tempered therapy and stability support of which are targeted to specific areas of need of which an individual may require at the time periods before, during, and after participating in an activity. The device includes two separate patella jump bands, one band for the upper knee area, and the other for the lower area of the knee. The center inner surface areas of the bands form into single or multiple pockets of which are specifically formed to receive therapeutic hot/cold tempered material. The outer surface layer of the formed pockets have a Velcro type of material affixed to their surface areas in order to allow separate individual pockets and pads to attach to it's lined surface as would be individually desired. Also comprising the device are two connecting left and right side bands of which serve as bridges to connect the upper and lower patella bands by attaching and joining together the patella bands to the left and right side connecting bridge bands in order to integrate and join the four separate components into one complete structural device. The inner layer surface areas of the two left and right side connecting bridge bands form into pocket enclosures of which are formed specifically to receive therapeutic hot/cold tempered material. The outer surface layers of the formed pockets have a Velcro material affixed to their surface areas in order to allow separate individual pockets and support pads to attach to its Velcro lined surface as would be individually desired. The upper and lower patella jump bands may be worn separately if desired; they may also be worn in conjunction with the connecting bridge bands for extra support if needed. Therapeutic hot/cold material may be added to the several strategically positioned designated areas throughout the areas of the separate device components, and thus providing for a much needed and convenient therapeutic device alternative to the wearer. Also the stability support components are readily available to attach to the several strategically positioned designated areas throughout the separate device components, and thus offering simultaneous optional stability and therapeutic control. Another separate integral component segment of the device includes a fold over flap piece of which attaches onto the surface of the device via Velcro. The fold over flap piece is specifically formed to position itself in such a manner as to substantially cover the center area of the knee once the flap has been appropriately secured onto the surface of the device. The entire inner surface layer area of the flap forms into single or multiple pockets of which are specifically formed to receive therapeutic hot/cold tempered material. The outer surface layers of the pockets are lined with Velcro in order to make it a possible option to attach separate individual pockets and support pads to the surface area.

### DETAILED DESCRIPTION

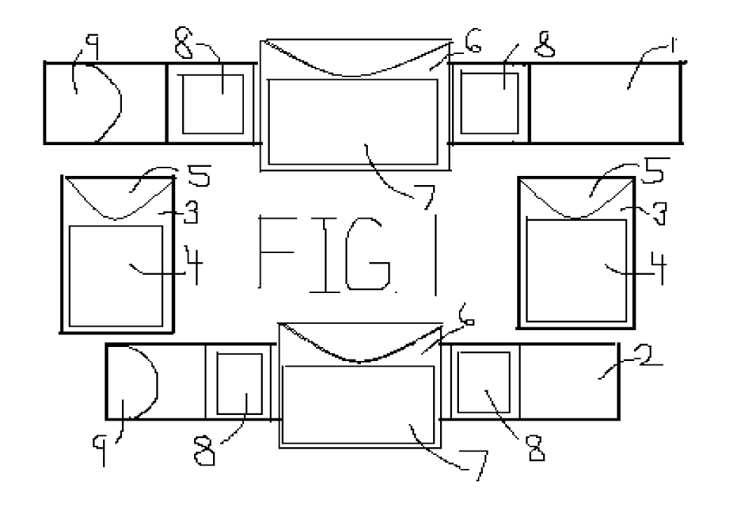

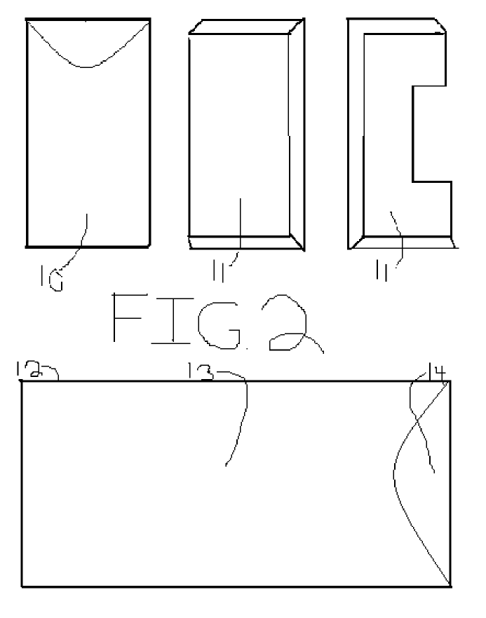

Referring now to the drawings and more particularly to FIG. 1. There is shown an inner layer perspective view of an embodiment of an interchangeable knee device manufactured utilizing the method of the present invention. In the embodiment shown the knee device consists of an upper patella band (1) of which is comprised of a neoprene or similar type of material thereof. The center portion inner layer surface area of the upper patella band consists of an overlapping piece of a neoprene foam type of material of which is positioned, cut and stitched in such a manner as to form a pocket enclosure, FIG. 1 (6) of which is specifically formed to receive a therapeutic hot/cold tempered material. The outer surface layer of the upper patella band's center located pocket enclosure, FIG. 1 (7) has a Velcro type of material affixed to its surface, as to allow the attachment of additional separate pocket enclosures, FIG. 2 (10), and or cushion support pads FIG. 2 (11) as explained in these specifications. FIG. 1 (8) illustrates the inner surface layer areas of the upper patella band in which Velcro is affixed to the surface areas as to allow the left and right side connecting bridge bands FIG. 1 (3), to attach to accordingly in order to properly assemble the device into its complete form. The upper patella band is purposely formed to be longer, wider and thicker in size and dimension than the lower patella band (2) as thus to fit proportionally around the circumference of its intended area of wear. FIG. 1 (2) consists of a lower patella band of which is comprised of a neoprene or similar type of material thereof. The center portion inner layer surface area of the lower patella band consists of an overlapping piece of a neoprene foam type of material of which is positioned, cut and stitched in such a manner as to form a pocket enclosure FIG. 1 (6) of which is specifically formed to receive a therapeutic hot/cold tempered material. The outer surface layer of the lower patella band's center located pocket enclosure FIG. 1 (7), has a Velcro type of material affixed to it's surface as to allow the attachment of additional separate individual pocket enclosures and cushion support pads FIG. 2 (10,11) as further explained in these specifications. The lower patella band has Velcro affixed to the left and right center areas (8) of the inner surface layer, as to allow the left and right side connecting bridge bands (3) to attach to the Velcro lined surface of (4), as will be further explained in these specifications. FIG. 1 (3) consists of left and right side connecting bridge bands of which act as bridges to connect and join the upper and lower patella bands together to form one complete unit structure. The left and right side connecting bridge bands (3) are comprised of a neoprene material. The inner surface layer area of each individual left and right connecting bridge band consists of an overlapping piece of a neoprene foam type of material of which is positioned, cut and stitched in such a manner as to form a pocket enclosure FIG. 1 (5) of which is specifically formed to receive a therapeutic hot/cold tempered material. The outer surface layer areas of the left and right side connecting bridge band's pocket enclosures (4), have a Velcro type of material affixed to it's surface so as to allow the attachment of additional pocket enclosures and cushion support pads FIG. 2 (10,11) as further explained in these specifications. The FIG. 1 (9) areas of the upper and lower patella bands consist of a Velcro type of material affixed onto it's inner surface layer so as to allow each individual band to fasten securely around the circumference of the designated area of the leg.

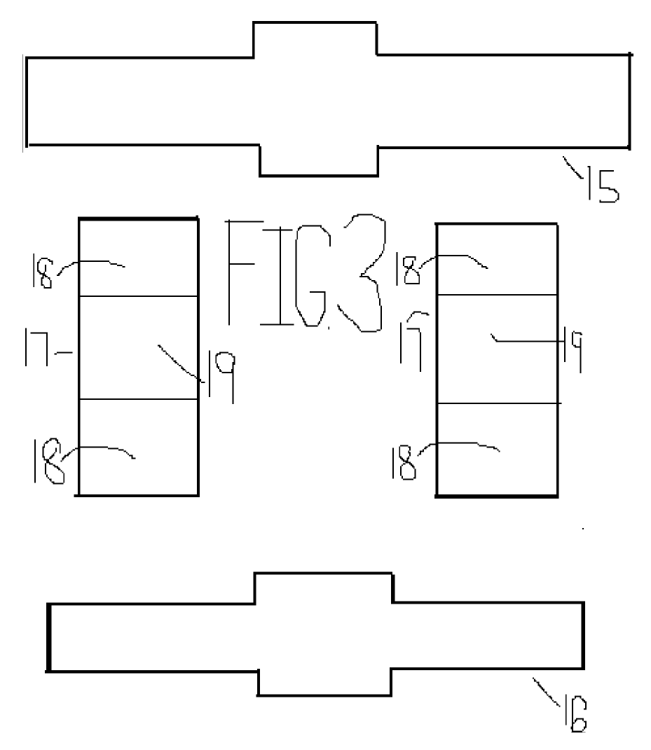

FIG. 2 is a perspective view illustrating the separate attachment pieces of which are comprised of individual pocket enclosures (10), individual cushion support pads (11), and a front fold over flap (12), of which integrate together with the pieces illustrated in FIG. 1 by attaching to one another via the method of Velcro adhesion. The separate attachment pieces illustrated as (10) consist of a pocket type enclosure comprised of a neoprene type of material. The pocket enclosure may be of any size and dimension as needed. The rear side outer layer surface area of the attachable/detachable pocket enclosure is affixed with a Velcro type of material of which allows the pocket to attach and detach to designated areas of FIG. 1 illustrated as (4,7) as would be individually needed via the method of Velcro adhesion. The attachable/detachable pocket enclosure illustrated as FIG. 2 (10) is specifically formed to receive a therapeutic hot/cold tempered material. FIG. 2 (11) illustrates separate individual cushion support pads of which are comprised of a neoprene foam type of material. The rear side outer layer surface area of the cushion support pad consists of a Velcro type of material affixed to it's surface so as to allow the pad to attach/detach via the method of Velcro as would be individually needed to the designated areas of FIG. 1 illustrated as (4,7). The cushion support pads may be of any individually desired size and dimension. FIG. 2 (12) is an inner layer perspective of an attachable fold over flap piece of which attaches onto the outer surface layer areas illustrated as FIG. 3 (15, 16, 19) as will be further explained in these specifications. A Velcro type of material is used to attach the fold over flap piece to the outer layer surface of the device. The fold over flap piece is specifically formed to position itself to substantially cover the center knee area once the piece is attached. The area illustrated as (12) is comprised of a neoprene/foam type of material of which it's inner layer area is specifically overlaid, positioned, cut and stitched in such a manner so as to form a pocket type enclosure (14), of which is specifically formed to receive a therapeutic hot/cold tempered material. The inner layer surface area of (12) is lined with a Velcro type of material of which spans and covers the entire inner surface layer area, and is specifically formed to allow separate single or multiple pocket type enclosures as illustrated in FIG. 2 (10) of which are specifically to contain therapeutic hot/cold tempered material of which would be inserted into it's open pocketed area and then having the pocket securely fasten to the inner surface layer area of the fold over flap piece, in order to provide therapeutic hot/cold tempered therapy to the intended knee area.

FIG. 3 illustrates an outer layer perspective view of the embodiment known as FIG. 1. FIG. 3 (15) illustrates the outer layer surface of the upper patella band. The outer layer consists of a Velcro type of adhesive material spanning throughout it's entire outer surface area, of which allows the attachment of the front fold over flap piece FIG. 2 (12), to the patella band's outer layer surface. Area (16) illustrates the outer layer surface of the lower patella band. The outer layer consists of a Velcro type of adhesive material spanning throughout its entire outer surface area of which allows the attachment of the front fold over flap piece FIG. 2 (12), to the patella band's outer layer surface. Area (17) illustrates the rear side view of the left and right side connecting bridge bands known as FIG. 1 (3). Affixed onto the surface areas illustrated as (18) is a Velcro material of which is strategically positioned to attach via the method of Velcro to the inner side layer areas known as FIG. 1 (8) of the upper and lower patella bands in order to assemble the basic structure of the device. Affixed onto the surface areas illustrated as (19) is a Velcro type of material of which is strategically positioned to attach to, via Velcro to the inner side layer areas of the front fold over flap piece known as FIG. 2 (13), in order to attach the flap onto the outer surface of the device.

What is claimed is:

1. An interchangeable knee support device of which incorporates the use of therapeutic hot/cold tempered material. The device possesses separate upper and lower patella, ACL/MCL support devices incorporating one single embodiment of which is comprised of four main separate segments of which attach and integrate together to create the structure of the device thereof,
Comprised of:
A.) a first elastomeric leg wrap band,
i) Wherein the band is positioned to substantially cover the upper patella area of the leg located directly above the kneecap. Wherein the composite material comprising the center inner layer portion of the band is positioned, overlaid, cut and stitched in such a manner as to form single or multiple pocket type enclosures of which form specifically to receive therapeutic hot/cold tempered material. ii.) A second elastomeric leg wrap band wherein the band is positioned to substantially cover the lower patella area of the leg directly below the kneecap, wherein the composite material comprising the center inner layer portion area of the band is positioned, overlaid, cut and stitched in such a manner as to form single or multiple pocket type enclosures of which form specifically to receive therapeutic hot/cold tempered material.
B.) Two interchangeable elastomeric bands of which are positioned vertically and parallel to each other, of which are positioned to the left and right sides of the device with the composite material of which comprises the inner layer surface area of the band is positioned, overlaid, cut and stitched in such a manner as to form single or multiple enclosures of which form specifically to receive therapeutic hot/cold tempered material. i.) the bands are used as bridges to connect the upper and lower patella leg wraps in order to create the attached structure of the device. ii.) the two elastomeric connecting bridge bands are positioned on the left and right sides of the device in such a manner as to allow coverage of the left and right outer side areas of the knee when the device is attached, worn, and fastened onto the knee area.

2. The device of claim 1 wherein the outer surface layer area of the formed pocket enclosures are affixed and lined with a Velcro type of material.

3. The device of claim 2 wherein therapeutic hot/cold tempered material is inserted into separate attachable individual pocket type enclosures of any desired size and dimension and are then attached the Velcro lined outer surface layer areas of the formed pockets.

4. The device of claim 2 wherein the pockets are specifically formed to receive therapeutic hot/cold tempered material.

5. The device of claim 1 wherein the formed and integrated pockets may be of any size and dimension.

6. The device of claim 2 wherein separate individual cushion support pads of any desired size and dimension may attach and or detach from the Velcro lined outer surface layers of the formed pockets.

7. The device of claim 1 wherein an attachable fold over flap is attached onto either the outer or inner Velcro lined surface layer areas of the device and is positioned in such a manner as to substantially cover the center area of the knee.

8. The device of claim 7 wherein the composite material comprising the inner surface layer area of the flap is positioned, overlaid, cut and stitched in such a manner as to form single or multiple pocket type enclosures of which form specifically to receive a therapeutic hot/cold tempered material.

9. The device of claim 8 wherein the outer surface layer area of the formed pocket enclosure(s) is affixed and lined with a Velcro type of material.

10. The device of claim 8 wherein therapeutic hot/cold tempered material is inserted into separate attachable, individual pocket type enclosure(s) of any size and dimension and are then attached onto the Velcro lined outer surface layer areas of the formed pocket(s) located on the inner surface layer area of the flap.

11. The device of claim 9 wherein separate individual cushion support pad(s) of any size and dimension may attach to the Velcro lined inner surface layer area of the flap.
